(19) United States
(12) Patent Application Publication
(10) Pub. No.: US 2018/0243499 A1
(43) Pub. Date: Aug. 30, 2018

(54) DISPOSABLE CONTAINER

(71) Applicant: Rotho Kunststoff AG, Würenlingen (CH)

(72) Inventors: Marco VISENTIN, Musile di Piave (IT); Marcel INÄBNIT, Würenlingen (CH)

(21) Appl. No.: 15/545,993

(22) PCT Filed: Jan. 22, 2016
(86) PCT No.: PCT/CH2016/000012

§ 371 Date: Jul. 24, 2017

(30) Foreign Application Priority Data
Jan. 23, 2015 (CH) 00083/15

(51) Int. Cl. A61M 5/00; B29C 49/48; A61B 17/3215

(52) U.S. Cl. CPC A61M 5/002; B29K 2067/003; A61B 17/3215; B29C 49/48

(57) ABSTRACT

A disposable container (1, 1′, 1″), in particular for the disposal of sharp medical waste is provided, comprising a first opening (13, 13′) sealingly closable by a cover element (2), characterized in that the container is manufactured by blow-moulding; the largest cross-sectional diagonal of the container in the distal half with respect to the first opening is larger than the smallest cross-sectional diagonal of the container in the proximal half with respect to the first opening; and the container comprises a second opening (15, 15′) arranged opposite to the first opening or in the distal area with respect to the first opening, the second opening being sealingly closable by a base element (11, 11′).

### FIELD OF THE INVENTION

The present invention relates to a disposable container, in particular for the application in a medical environment, for example in hospitals, laboratories, pharmacies or wherever medical waste has to be disposed.

### BACKGROUND OF THE INVENTION

Containers which are disposable can have a variety of applications in daily life, for example for the disposal of waste. In the case of waste material which could invoke safety issues when taken out of the container it is commonly desired if the container is disposable together with the waste as soon as the container is full.

Such disposable containers are preferably manufactured in a low-priced process using a cheap raw material. Many of the common containers are manufactured by injection-moulding using plastics, such as for example polypropylene (PP).

An example for the necessity of such disposable containers is for example given if the waste material comprises sharp devices with potential contaminations, for example in medical environments. An important application for disposable containers are sharps disposal containers for the disposal of sharp medical waste.

A disposable container can be desirable also for waste comprising non-sharp items such as cotton, catheters or the like due to contamination issues.

Commercially available sharps disposal containers commonly comprise a container body consisting of for example PP and produced for example by injection-moulding. The container body commonly comprises an opening through which the sharps are disposed and which is sealingly closable by a cover.

A collecting and waste disposal container is for example described in EP1380316 B1. The container has a bottom part and a lid comprising a feed aperture and a closing element provided in the arrangement of a supporting frame framing the feed aperture. The supporting frame and the closing element form a movable covering and closing unit for the feed aperture. An upper configuration is provided for the closing element for only temporarily covering the feed aperture and a lower configuration which can be adopted by the application of force from the upper position for the final covering thereof. The container has an elliptical cross section which widens to the top in a slightly conical manner.

A container for the disposal of hospital supplies including a parallelepiped element-type box is described in WO2008/106759 A1, which has, in its upper surface, a circular hole for disposal, and also provided in the upper portion a dumping cover, with a hinge on the back portion in material continuity with regards to the posterior face of the box, a system of locks or sealing, consisting of a rectangular projection containing a hole at its centre, the parallelepiped element and its cap produced in waterproof polymer material, resistant to puncture, rupture and leakage. The polymer used in the manufacture of the container is derived from recycled polyethylene terephthalate (PET).

U.S. Pat. No. 6,062,001 A describes a disposable container for safe disposal of used pen syringe needles including a plurality of walls defining a receptacle for receiving used needles therein. The assembled container functions as disposable storage for used needles from a pen syringe, particularly in a home healthcare environment. The container including the cover and the closure device are all molded by conventional plastic injection methods. The container is made of puncture resistant materials for safely storing used needles.

WO2007/130402 A2 describes a medical sharps and waste disposal container made from a composition of polypropylene and an additive having an impact strength sufficient to prevent the escape of sharps from the container. The additive is present in an amount sufficient to accelerate degradation of the polypropylene after a predetermined shelf-life.

WO90/14851 describes a cannula destructor with a container comprising a container casing which is open at both ends thereof, a bottom which functions as a removable cover member, and a cap which is detachably suspended on the underside of the bottom and which functions to close the upper open end of the container. The container casing is a one-piece structure and includes frustoconical sections, namely a base section, an intermediate section and a top section, with a conicity which increases in a direction away from the base section, a cylindrical neck portion, and a frustoconical top section, the base of which faces in a direction opposite to the casing sections and forms a circumferentially extending, protective collar which projects outwardly from the cylindrical neck portion.

Within the container there is provided in the proximity of the neck part a wall part which is formed integrally with the container casing and which completely covers the neck part internally. This wall has provided therein a large waste hole through which objects to be scrapped, such as used needles, cannula holders, etc. can be inserted and a cannula release mechanism including two keyhole-shaped locking devices which are located one above the other and which can be rotated relative to one another through 180°. This construction is not achieved by blow-moulding and furthermore the wall and its cannula release mechanism are disadvantageous in manufacturing and use.

EP0168877 B1 describes a container for the storage of sensitive products, for example, contaminated waste such as hospital refuse. The container has a base and joining thereupon standing wall members, wherein the open top bordered by the upper edges of the wall members can be closed by a cover whose edge member is carried by the upper edges of the standing wall members. Said edge member of the cover has a groove or channel with sealing means for accommodating the upper edges of the wall members, and the edge member and upper edges are provided with integral locking means for locking said cover on said container. The one of said means is formed by flexible tabs, each being provided with a first abutment surface. The other of said locking means is formed by a protruding rib provided with apertures for receiving said flexible tabs and a second abutment surface against which abut the first abutment surfaces when the cover is locked to the container.

### SUMMARY OF THE INVENTION

Due to their single-use character, there are several particular issues arising when manufacturing disposable containers. For example, any reduction of costs on the material side can be particularly important and can be realized for example by the choice of the appropriate raw material or by the optimization of the manufacturing process. Injection-moulding is a common process with which a large number of disposable containers can be manufactured efficiently.

Since such containers are usually supplied in large numbers, it is furthermore advantageous if the containers can be stacked such that the volume of the supply is minimized. Commonly, the containers comprise one opening through which the container is filled and through which the several containers of a stack are nested.

The commercial product, i.e. the assembled container, typically includes a set of all parts in connection with a disposable container. For the purpose of this description, disposable container, or container, is just referring to the main body part of such a commercial product. The present invention relates to such disposable containers and the combination of the container and its supplemental parts.

An issue arising from the injection-moulding process is that due to the requirement of releasing the container from the mould and the mandrel after injection-moulding and the fact that the opening of the container points upwards when positioning the container on ground, the centre of gravity of such containers usually lies in the upper half of the containers. Therefore, the stability against load is reduced and the containers are prone to tipping over.

The stability of the container is a particular aspect in the context of the general demand that the container should be designed such that the content of the container can not be released unintentionally.

A special attention should be given at the application of such containers in medical environments, such as hospitals, where the use of devices or device components with sharp edges or ends and/or contaminated material requires reliable precautionary measures to safeguard both the personnel and the patients against injuries and possible resulting infections.

In this respect, the safe disposal of such sharp and/or contaminated medical waste after their use represents a particular issue. An appropriate sharps disposal container for this purpose may fulfil several requirements such as for example sufficient puncture-resistance, stability under load, tightness for keeping the content inside the container against external influences such as shock, pressure, moisture, etc., identification means such as distinct labelling or colouring, etc. For the use of syringes, appropriate stripping arrangements for stripping the needles into the container may be additionally required.

An important aspect concerns secure and easy handling of sharps disposal containers. Means for secure handling should help to avoid possible injuries while using such sharps disposable containers. An appropriate geometry of the container can be advantageous for secure handling if for example the geometry provides stability against tipping over. The sharps disposal container should enable easy handling such that the possibility of faulty operation of the container possibly leading to injuries is reduced. Preferably, also a user not familiar with handling of medical devices should be able to use the sharps disposal container in a safe manner.

By manufacturing sharps disposal containers, it is necessary to choose the wall thickness of the container body sufficiently thick in order to ensure puncture-resistance. On the other hand, a reduction of material input can result in significant reduction of costs due to the large piece numbers usually produced in injection-moulding processes yielding the manufacture of thin walls desirable.

Furthermore, there is a demand for a certain transparency of the container body in order to have a means to recognize the filling level of the container and to prevent overloading which sets an upper limit to the wall thickness of the container body. Overloading of the container may hold the risk that a user may accidentally get in contact with some of the potentially harmful content of the container.

It is therefore an object of the present invention to modify and improve the state of the art of disposable containers, in particular for disposing sharp and/or contaminated medical waste complying safety issues, thereby modifying and improving flexibility and reducing material costs.

This object is achieved by the subject matter of the independent claims. Exemplary and/or favourable embodiments are further defined by the dependent claims and the disclosure of this document.

According to one aspect, the object is achieved by a disposable container, in particular for the disposal of sharp medical waste. The container comprises a first opening sealingly closable by a cover element. In general, the container is manufactured by blow-moulding. Furthermore, the largest cross-sectional diagonal of the container in the distal half with respect to the first opening is larger than the smallest cross-sectional diagonal of the container in the proximal half with respect to the first opening. In general, the container comprises a second opening arranged opposite to the first opening or in the distal area with respect to the first opening, the second opening being sealingly closable by a base element.

In the context of this document, “upwards”, “upper”, “top” is to be understood as the respective direction in relation to a normal working position. A working position may be achieved for example by vertical positioning of the container on a table. The expressions “lower”, “downwards”, “bottom” are to be understood accordingly.

The container is in general positioned in such a way that the first opening points upwards. Thus, the largest cross-sectional diagonal of the container in the lower half is larger than the smallest cross-sectional diagonal of the container in the upper half. For constant wall thickness, this generally results in the centre of gravity of the container lying in the lower half of the container. Since the centre of gravity of a body determines its stability against tipping over to a large extent, the low-lying centre of gravity of the container according to the present invention prevents said container from tipping over. The volume or the geometry of the container results in the centre of gravity being in the lower half of the container also if the container is filled.

In the context of this document, the cross-sectional diagonal is understood as a diagonal of a horizontal cross-section of a body. For a truncated cone with circular cross-section, the largest cross sectional diagonal is the diameter of the circular base of the cone. For a polygonal cross-section, a diagonal is understood as a line connecting two nonadjacent polygon vertices. For an ellipsoidal cross-section, the largest diagonal is the major axis of the ellipse. The largest cross-sectional diagonal of a body is understood as the largest diagonal of all largest diagonals of the cross-sections of said body.

In a preferred embodiment, the shape of the container is a truncated cone with the first opening located at the top plane surface. The surface of the first opening may be flush with the top plane surface. The area of the first opening may be smaller than the area of said top plane surface such that a circumferential collar is formed at said top plane surface of the truncated cone.

Conventional containers usually have one opening through which for example the container is filled. The container may be filled through the one opening through which for example waste is disposed. For such conventional injection-moulded containers with one opening, the opening of a for example frustoconical container needs to be situated at the plane surface with larger area in order to be able to release the container from the mould and the mandrel. This results in commercially available injection-moulded containers for the disposal of sharp medical waste having usually their centre of gravity in the upper half of the body reducing the stability against tipping over.

For the container according to the present invention, however, such a drawback is avoided by the geometry comprising two openings. For example, the second opening may originate from the moulding process and may correspond to the above mentioned one opening of a conventional container. In contrast to the conventional container where the one opening always has to be oriented upwards in order to be able to receive the waste, the container according to the present invention is free in orientation. For example, the container can be turned such that the second opening originating from the moulding process can be oriented downwards since the first opening on the opposite can serve as an opening to receive the waste. Therefore, the container can be turned such that the centre of gravity is positioned in the lower half of the container.

Preferably, the first opening and the second opening are arranged opposite to each other. In a typical rounded geometry of the container, such as for the embodiment where the container has the shape of a truncated cone, the opposite arrangement of the first and the second opening is understood as the openings being positioned at the top and the bottom plane surfaces of the truncated cone.

Preferably, the container is made by a blow-moulding process. Therefore, no mandrel is required and the container can be designed such that the first or the second opening of the container is situated within any desired surface of the container without limitations arising from the releasing process from the mould.

In a variation, the container may for example comprise a first opening at the top of the container and a second opening at a side face of the container. The first opening at the top may be used to fill the container. The second opening may be positioned at a side face in the bottom portion of the container. The second opening at the side face in the bottom portion of the container may be used to empty the container during the use. This may be particularly advantageous if the container is filled with a liquid content. Alternatively or in addition, the second opening at a side face of the container may be used as a sight opening to monitor the inner volume of the container.

The first opening of the container according to the present invention may originate from a core pin in a blow-moulding process through which air is blown into the preform or parison. Alternatively, the first opening may be cut out from the container after release of the container from the mould. Preferably, the cutting out of the first opening is achieved by punching. Excess plastic at the first opening may be trimmed after release of the container from the mould.

The first opening of the container may serve as an opening to receive the sharp medical waste such as syringe needles.

In order to prevent any content of the container from leaving the container, the first opening can be directly or indirectly sealingly closed by the cover element.

In general, the second opening is situated at a bottom plane surface of the container opposite to the first opening. The second opening may originate from the preform or parison in a blow-moulding process. For example, it may originate from a core pin in a blow-moulding process through which air is blown into the preform or parison. It may also originate from an injection-moulding process that generates the preform. Alternatively, the second opening may be cut out from the container after release of the container from the mould. The cutting out of the second opening can be achieved by punching. The second opening may be flush with the bottom plane surface of the container. The area of the second opening may be smaller than the area of the bottom plane surface. In a preferred embodiment, the complete bottom plane surface is recessed to form the second opening. Excess plastic at the second opening may be trimmed after release of the container from the mould.

The shape of the container according to the present invention with its first opening and second opening is particularly advantageous for stacking. Properties of containers which lead to a small volume when stacked, are advantageous and desired for supply purposes. The container according to the present invention may be stacked by nesting through the second opening which is generally situated at the bottom plane surface opposite to the first opening. After releasing a container from the stack, the second opening can be sealingly closed by the base element. The container can then be used with the first opening oriented to the top while the container maintains its stability against tipping over due to the largest cross-sectional diagonal of the container in the lower half being larger than the smallest cross-sectional diagonal of the container in the upper half.

Conventional sharps disposal containers with one opening can also be stacked by nesting through said one opening. However, due to the mentioned releasing issues from the injection-moulding process, the top surface of such conventional containers comprising said one opening has a larger diagonal than the bottom surface yielding the container instable against tipping over.

Thus, the container according to the present invention provides a stackable container and avoids at the same time instability issues.

A preferred blow-mouldable material which may be used for the container according to the present invention, is polyethylene terephthalate (PET). Compared to containers made from PP, PET has the advantage that the puncture-resistance of the container walls is higher for the same wall thickness. Thus, material costs could be reduced significantly. Furthermore, transparency of the container for the indication of the filling level can easily be achieved with PET while maintaining a wall thickness of the container for sufficient puncture-resistance.

The base element may be obtained by injection-moulding. The material of the base element may be PP. For safety issues, it is desired that the second opening is sealingly closable by the base element such that the content of the container can not be released. The sealingly closing may be achieved by a thread arranged at the container adjacent to the second opening and a counter thread arranged in the base element.

In a variation, the base element may be integrally formed with the container by blow-moulding such that the second opening is completely sealed and preferably permanently locked. Such a container with closed second opening and the first opening situated at the top plane surface with smaller diagonal than the bottom plane surface can be easily produced by blow-moulding due to the lacking need of a mandrel. However, for such a variation, nesting may not be possible.

Other complex geometries of the container with the largest cross-sectional diagonal of the lower half being larger than the smallest cross-sectional diagonal of the upper half of the container are possible. For example, the lower half of the container could have a polygonal cross section whereas the upper half of the container could have a circular or elliptical cross section. In another example, the lower half of the container could have an elliptical cross section whereas the upper half of the container could have a circular cross section. In yet another example, the container could be polygonal with the largest cross-sectional diagonal of the lower half being larger than the smallest cross-sectional diagonal of the upper half. It is understood by the one skilled in the art that other geometrical combinations are possible. Preferably, the centre of gravity remains in the distal half of the container with respect to the first opening also for other complex geometries.

In an embodiment, the container comprises at least one locking means, adapted for locking the cover element and/or the base element to the container. The at least one locking means may be integrally formed with the container. Alternatively or in addition, the at least one locking means may be a separate locking unit operatively interconnectable with the container.

In an embodiment, a first locking means is provided at the first opening, the first locking means comprising a locking ring, and a second locking means is provided at the second opening.

The at least one locking means may comprise a locking ring. The locking ring may be a separate unit operatively interconnectable with the container. Furthermore, the locking ring may be operatively interconnectable with the cover element. The locking ring may comprise recesses which are operatively interconnectable with pins of the cover element. Alternatively or in addition, the locking ring may comprise pins which are operatively interconnectable with recesses of the cover element. In a variation, the locking ring may comprise a thread which is operatively interconnectable to a thread of the cover element. Optionally, the locking ring may comprise bayonet joint elements.

In an embodiment, the at least one locking means comprises a thread.

In an embodiment, the at least one locking means comprises bayonet joint elements.

In an embodiment, the at least one locking means comprises latches.

Alternatively or in addition, the at least one locking means comprises recesses.

Preferably, the container is operatively interconnectable with the cover element by one of the at least one locking means. The one of the at least one locking means may be positioned next to the first opening.

In embodiments comprising a collar next to the first or the second opening, the at least one locking means may comprise recesses within the collar.

Alternatively or in addition, the container is unreleasably interconnectable with the cover element in a force-fit manner. Alternatively or in addition, the container is unreleasably interconnectable with the cover element in a form-fit manner.

It may be advantageous to unreleasably interconnect the container with the cover element due to safety issues, in particular, to avoid accidental removing of the cover element sealingly closing the first opening of the container.

In an embodiment, the container comprises at least one circumferential stabilization bead. The stabilization bead may support the stability of the container at critical spots where the stability given by the wall thickness is not sufficient. This may be the case near an opening of the container. For example, for the first opening of the container with locking means comprising a thread and the thread being situated next to the first opening, a stabilization bead near the first opening can provide sufficient torsional stability when engaging the thread.

In an embodiment, the container comprises a base element, the second opening being closable by said base element, and said base element comprising a circumferential outer side wall and a circumferential inner side wall. The portion of the container adjacent to the second opening may be receivable in the space between the outer and inner side wall. The configuration with the outer side wall and the inner side wall may be advantageous to improve the stability of the base element. Furthermore, the outer side wall and the inner side wall may be configured to improve the sealed closing of the second opening by the base element when the portion of the container adjacent to the second opening is received in the space between the outer and the inner side wall. The inner or the outer side wall of the base element may comprise a thread operatively interconnectable to a thread at the portion of the container adjacent to the second opening.

In an embodiment, the container comprises a single-walled base element. The single wall of the base element may comprise a thread operatively interconnectable to a thread at the portion of the container adjacent to the second opening.

Preferably, the container is operatively interconnectable with the base element by one of the at least one locking means. The one of the at least one locking means may be positioned next to the second opening. Said one of the at least one locking means may be a thread which may be operatively interconnectable with a counter thread of the base element. Said operative interconnection may be a unreleasable or a releasable interconnection. In a variation, the releasable interconnection may be only releasable above a minimum applied force.

Alternatively or in addition, the container is unreleasably interconnectable with the base element in a force-fit manner. Alternatively or in addition, the container is unreleasably interconnectable with the base element in a form-fit manner. It may be advantageous to unreleasably interconnect the container with the base element due to safety issues, in particular, to avoid accidental removing of the base element sealingly closing the second opening of the container.

In an embodiment, the container may comprise a stabilization bead next to the second opening. For example, for the second opening of the container with locking means comprising a thread and the thread being situated next to the second opening, a stabilization bead near the second opening can provide sufficient torsional stability when engaging the thread.

Preferably, the base element comprises counter base locking means operatively interconnectable to one of the at least one locking means of the container.

In an embodiment, the counter base locking means comprises a thread.

In an embodiment, the counter base locking means comprises bayonet joint elements.

In an embodiment, the counter base locking means comprises latches.

Alternatively or in addition, the counter base locking means comprises recesses.

In a preferred embodiment, the container is at least partially puncture-resistant.

The requirements on a sufficient puncture-resistance for sharps disposal containers are defined for example in DIN EN ISO 23907. According to the conformity test TRBA 250 a sufficient puncture-resistance is given by a resistance to a puncture of 15 N at a needle diameter of 1.1 mm with a BL/LB polished thin section. Preferably, the container according to the present invention may comply with said requirements.

In a preferred embodiment, the base element is at least partially puncture-resistant.

The person skilled in the art understands that in general it is sufficient to design those parts to be puncture-resistant which are in communication with the inner volume of the container. Selected other parts such as protection collars are preferably also designed to be puncture-resistant.

According to a further aspect, the object is achieved by a cover element for a container as disclosed in this document. Preferably, the cover element comprises a cover opening and an operatively interconnected cap element. The cover opening may be sealingly closable by the cap element. Furthermore, the cover element may be configured to sealingly close an opening of the container. The cover element may comprise an integrally formed circumferential hand protection collar protruding with a positive collar angle with respect to a horizontal face of the container.

Preferably, the cover element is configured to directly or indirectly sealingly close the first opening of the container. For safety issues, it is desired that the first opening is sealingly closable by the cover element such that the content of the container can not be released.

The cover element may directly sealingly close the first opening of the container in an embodiment where the cap element is integrally formed with the cover element such that the cover opening of the cover element is permanently sealed.

In an embodiment where the cover element comprises an operatively interconnected cap element, the cover element may indirectly close the first opening of the container by the cap element.

The cover element may be obtained by injection-moulding. The material of the cover element may be PP.

Preferably, the cover element is at least partially puncture-resistant.

The sharp medical waste may be disposed through the cover opening. The cover opening being sealingly closable by the cap element has the advantage that the container can be opened only when sharp medical waste is disposed and otherwise remain safely closed by the cap element.

Preferably, the cover opening may be flush with the first opening of the container.

Normally, the sharp medical waste such as a syringe needle or a scalpel blade is disposed using one hand holding the waste whereas the other hand is holding the container. The circumferential protection collar has the advantage that the hand holding the container is protected from eventual puncture by the sharp waste if the cover opening is not hit by the user disposing the sharp waste. The particular orientation of the protection collar having a positive collar angle with respect to a horizontal face of the container prevents slipping off the collar in the direction away from the cover opening when for example a syringe needle hits the collar.

Optionally, the cover opening comprises stripper elements for stripping syringe needles or all kinds of sharps, blades etc. The stripper elements may comprise protrusions and recesses. The protrusions and recesses may be designed such that for example needles of different syringe sizes can be stripped. The stripper elements may be designed that also other kind of items besides sharps can be disposed in the container using the stripper elements.

In an embodiment, the circumferential hand protection collar is foldable to a configuration with an inverted collar angle with respect to a horizontal face of the container. The configuration with an inverted collar angle reduces the stacking height of the cover element which may be advantageous for supply purposes. For example, the stacking height for stacking cover elements or stacking cover elements together with base elements or with containers may be reduced. The configuration of the protection collar with the positive collar angle with respect to a horizontal face of the container is the normal working position of the protection collar. For stacking or nesting, the protection collar may be folded to the configuration with the inverted collar angle. For the embodiment of the container with the shape of a truncated cone, a horizontal face with respect to which the collar angle is referenced to may be the top plane surface of the truncated cone.

Optionally, the cover element comprises a handle. The handle may be used to grab or hang up the cover element together with the container. In a variation, the handle is integrally formed with the protection collar.

In an embodiment, the cover element comprises counter cover locking means operatively interconnectable to locking means of the container.

In an embodiment, the counter cover locking means comprises a thread.

In an embodiment, the counter cover locking means comprises bayonet joint elements.

In an embodiment, the counter cover locking means comprises latches.

Alternatively or in addition, the counter cover locking means comprises recesses.

In an embodiment, the cover element is operatively interconnected to the container in a form-fit manner. The operative interconnection may be achieved by a two-step moulding process. For example, the injection-moulded cover element can comprise latches around which the container is blow-moulded such that the cover element is fixed in recesses of the container formed around the latches of the cover element during the blow-moulding process. Other two-step moulding processes to operatively interconnect the cover element and the container are possible. Operative interconnection in such a way is applicable also for the base element and the container.

Preferably, the cover element comprises cap locking elements, said cap locking elements being configured to unreleasably lock the cap element in a position seal-ingly closing the cover opening. It may be advantageous to unreleasably lock the cap element in the position sealingly closing the cover opening due to safety issues, in particular, to definitively close the full container and to avoid overfilling.

The cap element may sealingly close the cover opening of the cover element in two configurations: A first configuration where the cover opening is sealingly closed but can be reopened for disposing waste and a second configuration where the cap element is locked in the position closing the cover opening and can not be reopened.

In an embodiment, the cap locking elements comprise mutually mating pins and holes. The mutually mating pins and holes may engage with each other in the second configuration where the cap element is locked in the position closing the cover opening. The second configuration may be reached by pushing the cap element in the position closing the cover opening down towards the first opening.

In a variation, the cap locking elements comprise mutually mating latches and recesses. The mutually mating latches and recesses may engage with each other in the second configuration where the cap element is locked in the position closing the cover opening. The second configuration may be reached by pushing the cap element in the position closing the cover opening down towards the first opening.

Some of the cap locking elements may be integrally formed with the cap element. In a variation, some of the cap locking elements may be attached to the cap element.

In a variation, the cap locking elements comprise bayonet joint elements. The bayonet joint elements may engage with each other in the second configuration where the cap element is locked in the position closing the cover opening.

The cap element may be operatively interconnected to the cover element by various means. For example, a hinge may be foreseen operatively interconnecting the cover element and the cap element. Another example could be a cordrecess interconnection where the cord may be integrally formed with the cap element and the recess may be formed within the protection collar of the cover element. The cap element may, in a variation, interconnected by means of a cord in any other way known by the one skilled in the art. Other interconnection means known by the one skilled in the art are possible.

In an embodiment where the cap element is operatively interconnected to the cover element by a cord interconnection, the cord may be configured that the cord is torn apart when the cap element is brought into the second configuration where the cap element is locked in the position closing the cover opening. This may be advantageous since the second configuration can not be accidentally achieved without the cord being torn. Furthermore, a user can easily recognize that the second configuration is achieved by looking at the cord being torn.

The cap element may be made by injection-moulding. The material of the cap element may be PP.

In an embodiment, the cover element comprises a neck which can be received in the first opening of the container, preferably in a form-fit manner. The neck may support stable interconnection of the cover element with the container.

Preferably, the neck may run around the cover opening.

Optionally, the neck comprises pins which are operatively interconnectable with recesses of the locking ring. In a variation, the pins of the neck may be operatively interconnectable with bayonet joint elements of the locking ring. Alternatively or in addition, the neck comprises recesses which are operatively interconnectable with pins of the locking ring. In a variation, the neck comprises a thread.

The neck of the cover element may be receivable in the opening of the locking ring in a form-fit manner. Furthermore, the cover element and the locking ring may be operatively interconnectable in a configuration where the cover element is locked to the container. In embodiments where the top plane surface of the container comprises a circumferential collar, the collar may be pinched between the locking ring and a surface of the cover element in said configuration.

According to a further object, the object is achieved by a method of producing a container as disclosed in this document, wherein the container is produced by a blow-moulding process leaving the second opening open.

In an embodiment, the method of producing the container may be characterized in that in a second step the first opening is introduced by punching.

In an embodiment, the method of producing the container may comprise the base element being produced by injection-moulding.

The blow-moulding process may include injection blow-moulding, extrusion blow-moulding, stretch blow-moulding or other blow-moulding processes.

According to a further object, the object is achieved by a method of producing a cover element as disclosed in this document, wherein the cover element is produced by injection-moulding.

### LIST OF FIGURES

Embodiments of the invention will be more fully understood from the detailed description given herein below and the accompanying drawings, which should not be considered limiting to the invention described in the appended claims. The drawings are showing:

FIG. 1 a perspective view of an embodiment of a sharps disposal container comprising a base element, and a cover element comprising a cap element, the cap element closing a cover opening of the cover element;

FIG. 2 a perspective view of the sharps disposal container according to FIG. 1 with the cap element in a position where the cover opening of the cover element is open;

FIG. 3 an exploded view of the sharps disposal container and the cover element according to FIG. 1;

FIG. 4 the cover element, a locking ring and the container according to FIG. 3, the container shown in a cut view;

FIG. 5 the cover element, the locking ring and the container according to FIG. 4, with the locking ring operatively interconnected with the cover element;

FIG. 6 the cover element, the locking ring and the container according to FIG. 5, with the locking ring in a locked configuration;

FIG. 7 the cover element with the cap element according to FIG. 1 in a top view;

FIG. 8 a cut view of the cover element with the cap element along line A-A in FIG. 7;

FIG. 9 the cover element with the cap element according to FIG. 7, but with the cap element in a locked configuration;

FIG. 10 a cut view of the cover element with the cap element along line B-B in FIG. 9;

FIG. 11 a magnification view of the circle marked by C in FIG. 10;

FIG. 12 a perspective view of another embodiment of the container;

FIG. 13a a perspective view of yet another embodiment of the container;

FIG. 13b a side view of the container according to FIG. 13a;

FIG. 14a a perspective view of an embodiment of a base element;

FIG. 14b a side cut view of the base element according to FIG. 14a;

FIG. 15a a side view of an embodiment of the container with a protection collar in a normal working position;

FIG. 15b a side view of the container according to FIG. 15a with the protection collar folded with an inverted collar angle.

### DETAILED DESCRIPTION OF EXEMPLARY EMBODIMENTS

The present invention is better understood when read in conjunction with the figures enclosed. For the purposes of illustrating the invention, an embodiment that is presently preferred, is discussed in more detail with additional reference to the figures.

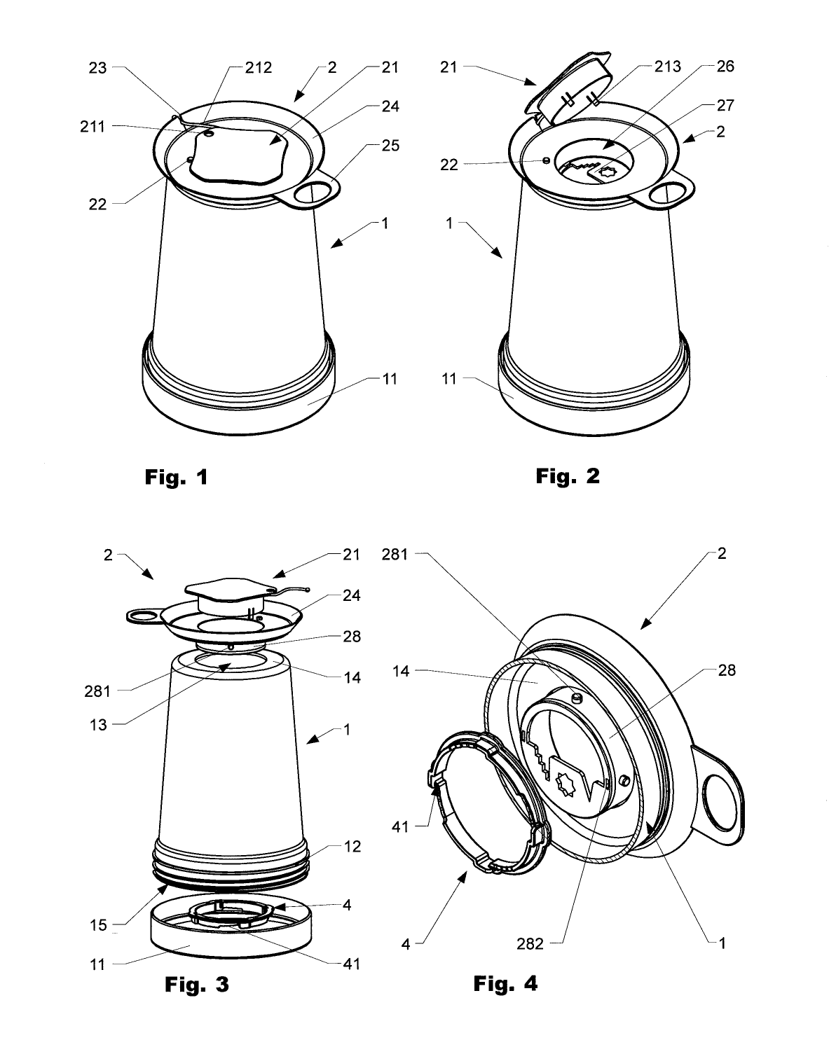

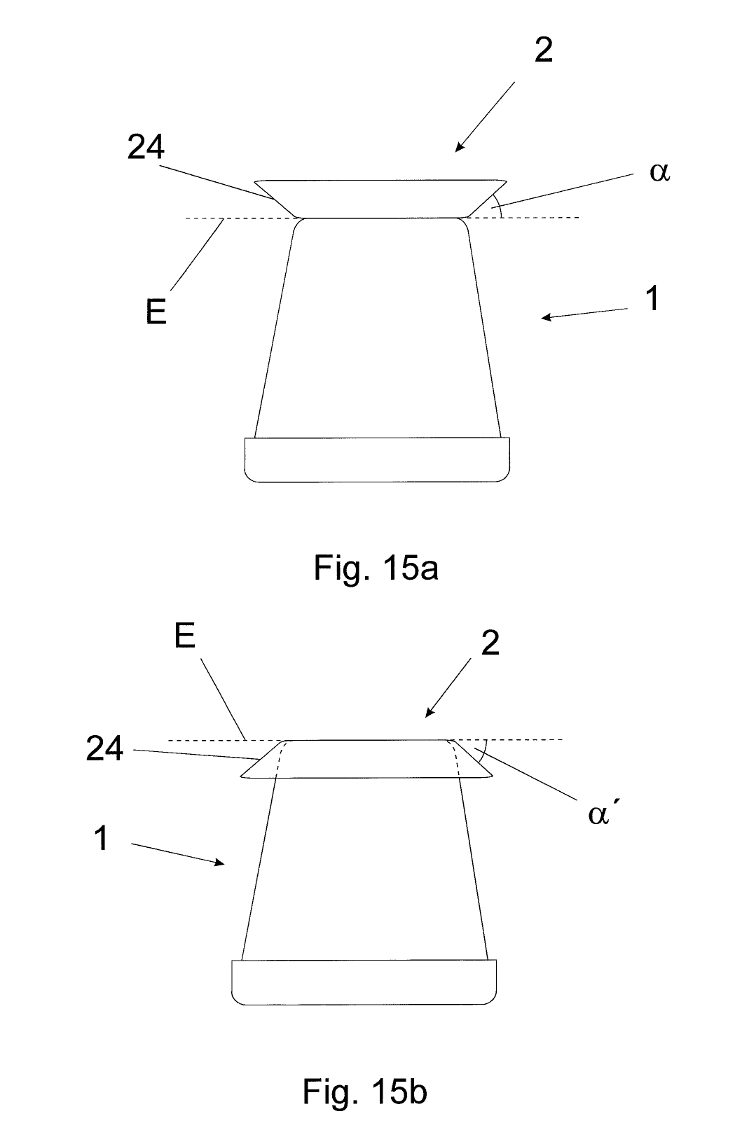

FIG. 1 shows a perspective view of an embodiment of a sharps disposal container 1, a base element 11, and a cover element 2 comprising a cap element 21. The shown container 1 has a shape of a truncated cone with its largest lower cross-sectional diagonal being larger than its smallest upper cross-sectional diagonal. In the shown embodiment of a truncated cone, the largest lower cross-sectional diagonal is the diameter of the circular base of the container 1 and the smallest upper cross-sectional diagonal is the diameter of the circular top surface of the container 1. Therefore, the centre of gravity is located in the lower half of the container 1 for an essentially constant wall thickness yielding an improved stability against tipping over. The base element 11 is shown sealingly closing a second opening (cf. FIG. 3) of the container 1. The cover element 2 preferably sealingly closes a first opening (cf. FIG. 3) of the container 1. The cap element 21 is shown in a first configuration closing a cover opening (cf. FIG. 2) of the cover element 2. The cap element 21 is operatively intero connected to the cover element 2 by a cord 212 and a cord recess 23. Other means for interconnecting the cap element 21 to the cover element 2 are possible. The cord recess 23 is shown being formed within a circumferential protection collar 24. The circumferential protection collar 24 forms a positive collar angle with respect to a horizontal face, for example the circular top surface, of the container 1 and is thus oriented upwards, as shown in the Figure (cf. also FIGS. 15a and 15b). In this way, a hand grabbing the container 1 may be protected from a sharp device such as a syringe needle approaching from the top by the protection collar 24. Cap locking means in the form of a pin 22 comprised in the cover element 2 and a hole 211 formed in the cap element 21 are shown not engaged. Therefore, the cap element 21 is shown in a first configuration closing the first opening of the container 1 in sealing manner, but where the first opening can be reopened.

Preferably, the container 1 is made by blow-moulding. The container 1, the cover element 2 and the base element 11 may be at least partially puncture-resistant. Those parts, which are in direct communication with the inner volume of the container 1, are puncture-resistant. Furthermore, the protection collar 24 is puncture-resistant (In certain embodiments, the collar 24 does not have the same puncture-resistance as the other parts). Preferably, the blow-moulded container 1, made for example of PET, has a wall thickness below 1 mm while maintaining a sufficient puncture-resistance. The container 1 according to the shown embodiment complies with the requirements as laid down for example in DIN EN ISO 23907 regarding for example the puncture-resistance.

The shown container 1 is made from PET. The shown cover element 2 and the base element 11 consist of PP and are made by injection-moulding.

FIG. 2 shows the sharps disposal container 1 according to FIG. 1 with the cap element 21 in a position where the cover opening 26 of the cover element 2 is open. The shown embodiment is an example where the cover element 2 can indirectly sealingly close the first opening of the container 1 by the cap element 21. Sharp medical waste can be disposed into the container 1 through the cover opening 26. In the shown embodiment, the cover opening 26 comprises stripper elements 27 for stripping syringe needles. The shown stripper elements 27 comprise protrusions and recesses which provide the possibility to strip needles of syringes of different dimensions. The protrusions and recesses are arranged as a terrace with gradations. In the shown embodiment, the stripper elements 27 further comprise a star-shaped recess for stripping needles. Latches 213 for locking the cap element 21 in a second configuration where the cap element is locked in a position closing the cover opening 26 may be integrally formed with the cap element 21, as shown in the Figure.

FIG. 3 shows an exploded view of the sharps disposal container 1 and the cover element 2 according to FIG. 1. The shown cover element 2 comprises a neck 28 which can be received in the first opening 13 of the container 1. The first opening 13 is formed after release from the mould by cutting. Preferably, the cutting out of the first opening 13 is achieved by punching. The cross-sectional area formed by the neck 28 preferably matches the area of the first opening 13. In the shown embodiment, the neck 28 comprises pins 281 which can engage with bayonet joint elements 41 of a locking ring 4 to operatively interconnect the locking ring 4 with the cover element 2. The area of the first opening 13 is smaller than the top plane surface of the container 1 such that a circumferential collar 14 is formed on which the cover element 2 abuts when sealingly closing the first opening 13 of the container 1. The shown container 1 comprises a thread 12 on its bottom portion by means of which the second opening 15 at the bottom plane surface of the container 1 can be sealingly closed by engaging with the base element 11. The thread 12 can releasably engage with a counter thread of the base element 11. Thus, in the shown embodiment, one of the at least one locking means of the container 1 is the thread 12 and the counter base locking means is the counter thread of the base element 11. In order to prevent accidental releasing of the thread 12, a minimum force may be necessary for releasing the thread 12. The thread 12 may be combined with latches such that container 1 can be unreleasably interconnected to the base element 11. The shown thread 12 has windings with a rather small slope such that a flat screwing with the base element 11 is possible. In a variation, a combination of the thread 12 with latches may be possible. The cover element 2 is shown in a configuration where the circumferential protection collar 24 forms a positive collar angle with respect to a horizontal face, for example the circular top surface, of the container 1 and is thus oriented upwards, as shown in the Figure. The protection collar 24 may have elastic features such that it is foldable to a configuration forming an inverted collar angle with respect to a horizontal face, for example the circular top surface, of the container 1 (cf. FIG. 15b). In the folded configuration, the collar 24 may be nested with the base element 11 yielding a small stacking height, which may be advantageous for supply purposes. In the shown embodiment, the complete bottom plane surface of the container 1 is recessed to form the second opening 15 of the container 1. The second opening 15 is formed by means of the pin blowing air into the preform or parison in a blow-moulding process or originates from an injection-moulding process that generates the preform. Containers in the shown shape may easily be stacked for supply purposes by nesting through the second opening 15.

FIG. 4 shows the cover element 2, a locking ring 4 and the container 1 according to FIG. 3. The container 1 is shown in a cut view, such that only the upper part comprising the collar 14 is visible. The locking ring 4 is shown in a configuration before engaging the bayonet joint elements 41 with the pins 281 of the neck 28. The area of the locking ring 4 preferably matches the cross-sectional area formed by the neck 28. The neck 28 comprises recesses 282 into which the latches 213 of the cap element 21 as shown in FIG. 2 can engage in order to reach the second configuration where the cap element 21 is locked in the position closing the cover opening 26.

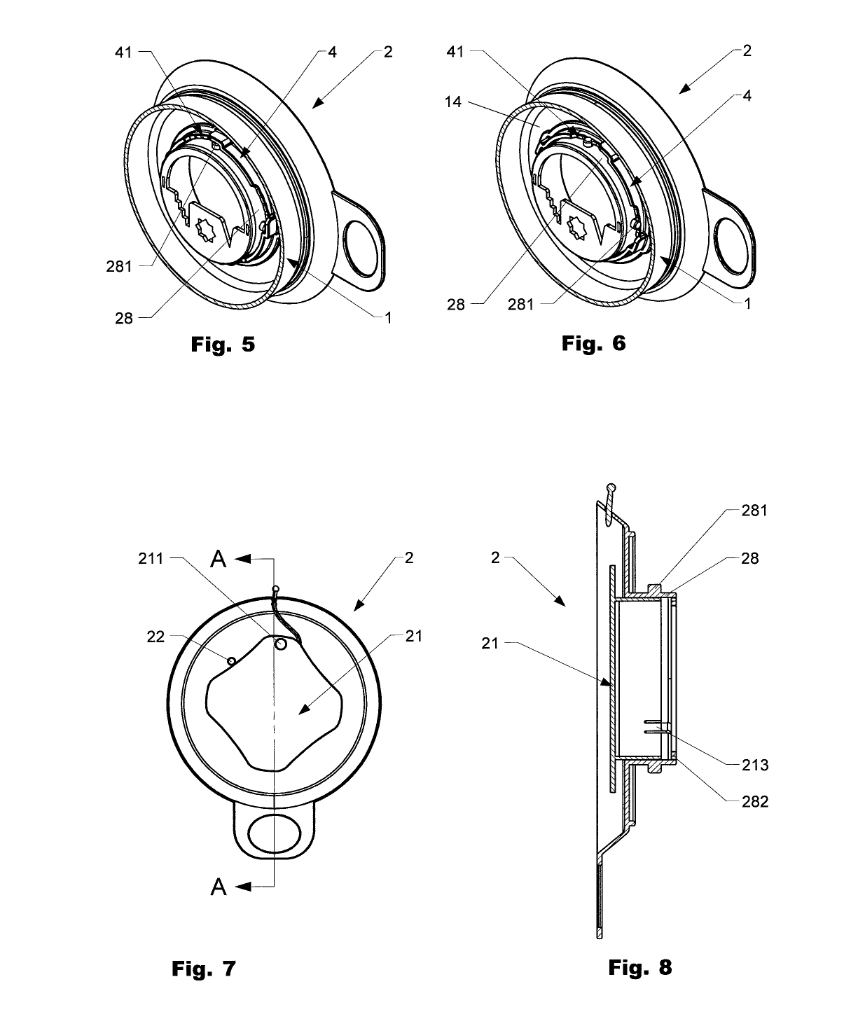

FIG. 5 shows the cover element 2, the locking ring 4 and the container 1 according to FIG. 4, with the locking ring 4 operatively interconnected to the cover element 2. The locking ring 4 is shown in a configuration where the bayonet joint elements 41 are engaged with the pins 281 of the neck 28, but not locked.

FIG. 6 shows the cover element 2, the locking ring 4 and the container 1 according to FIG. 5, but with the locking ring 4 in a configuration where the bayonet joint elements 41 are locked with the pins 281 of the neck 28. In the shown configuration, the cover element 2 is thereby locked to the container 1. The collar 14 is thereby pinched between the locking ring 4 and the cover element 2.

FIG. 7 shows the cover element 2 with the cap element 21 according to FIG. 1 in a top view. The cap element 21 is shown in the first configuration closing the cover opening of the cover element 2. The pin 22 is shown not engaged with the hole 211 such that the first opening can be reopened.

FIG. 8 shows a cut view of the cover element 2 with the cap element 21 along line A-A in FIG. 7. The cap element 21 is shown in the first configuration. In the shown configuration, the latches 213 of the cap element 21 are not engaged with the recesses 282 of the neck 28.

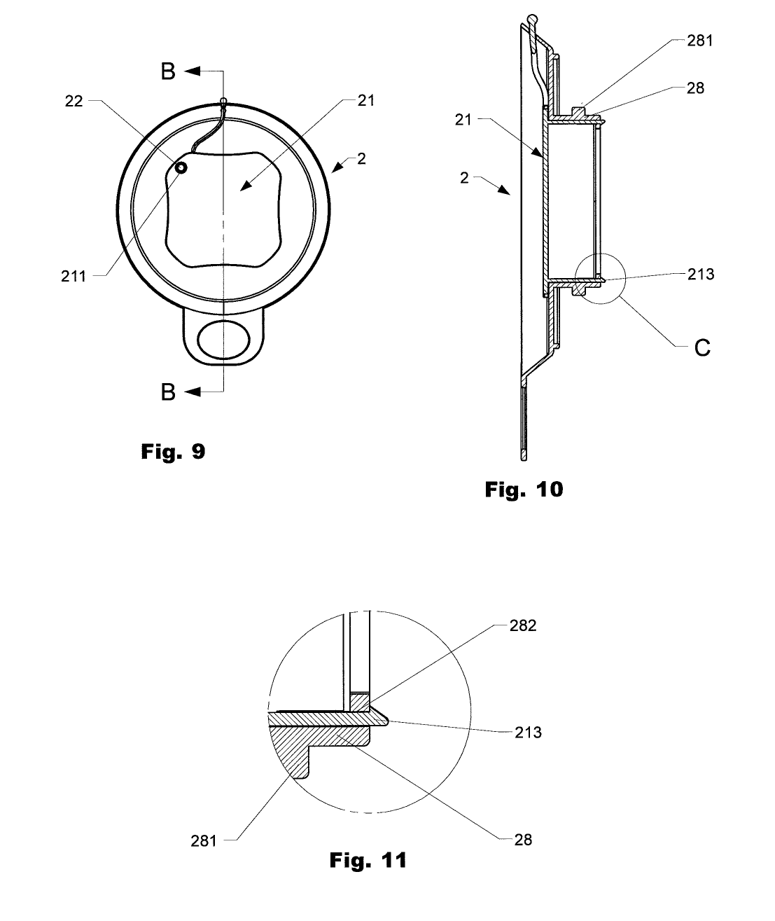

FIG. 9 shows the cover element 2 with the cap element 21 according to FIG. 7, but with the cap element 21 in the second configuration where the cap element 21 is locked in the position sealingly closing the cover opening. In the shown configuration, the pin 22 is engaged with the hole 211. In this way, the second configuration where the cap element 21 is locked may be visible and a user can recognize that the container 1 is full.

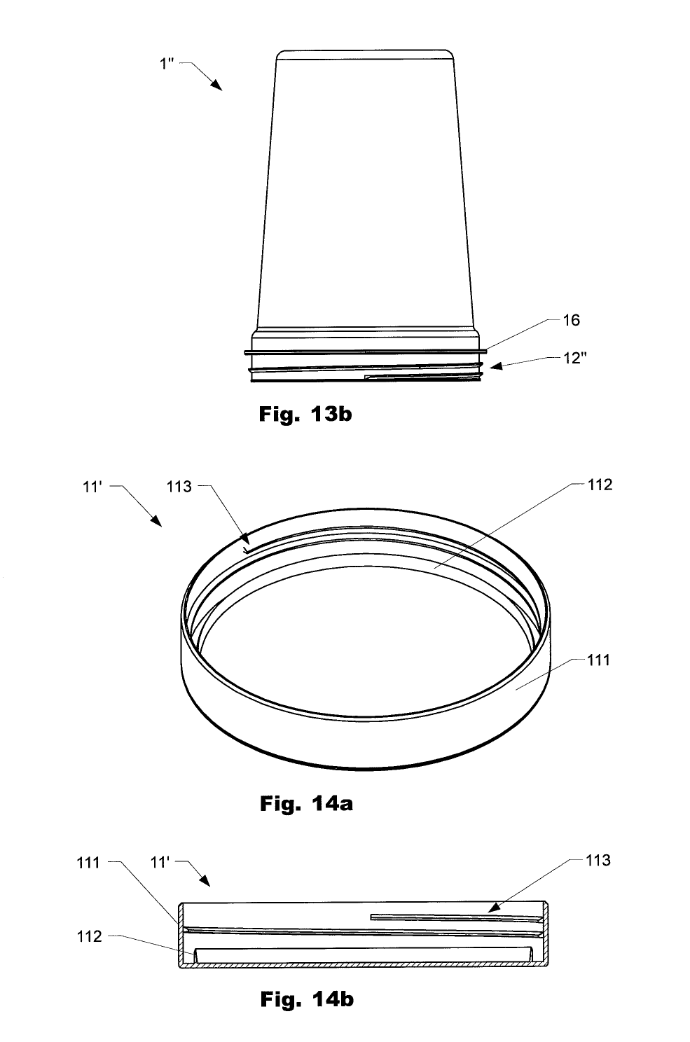

FIG. 10 shows a cut view of the cover element 2 with the cap element 21 along line B-B in FIG. 9. The cap element 21 is shown in the second configuration where the cap element 21 is locked in the position sealingly closing the cover opening 26. Compared to the first configuration as shown in FIG. 8, the cap element 21 is shown pushed down towards the cover element 2. Furthermore, the latches 213 are shown engaged with the recesses 282 in a latching manner such that the cap element 21 can not be removed and the cover opening 26 can not be opened. This configuration may be used as soon as the container 1 is full and can be disposed entirely. The second configuration can therefore be achieved by pushing the cap element 21 down towards the cover element 2 in a position where both the hole 211 engages with the pin 22 and the latches 213 engage with the recesses 282. The pins 281 are foreseen to engage with locking means (locking ring 4 according to FIG. 4 or inner neck 17 according to FIG. 13a).

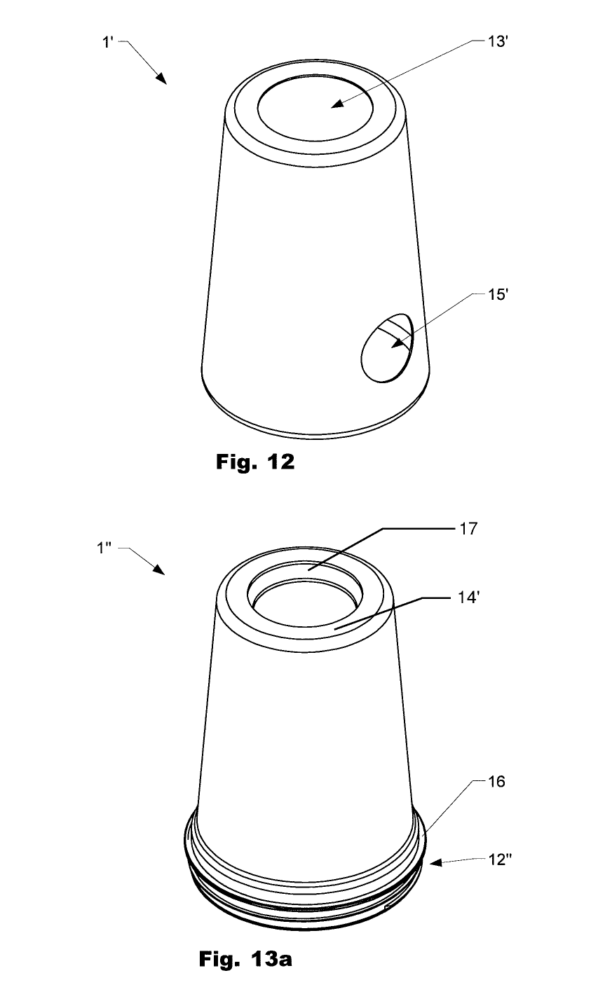

FIG. 11 shows a magnification view of the circle marked by C in FIG. 10. It can be recognized that the latch 213 of the neck 28 is engaged with the recess 282 in a latching manner.

In the embodiment as shown for example in FIG. 3, the first opening 13 and the second opening are arranged opposite to each other. The second opening is arranged at the bottom circular base of the container 1 whereas the first opening 13 is arranged at the top surface of the container 1.

FIG. 12 shows a perspective view of another embodiment of a container 1′ with a first opening 13′ and a second opening 15′. The first opening 13′ is positioned at the top of the container whereas the second opening 15′ is positioned at a side face in the bottom portion of the container 1′. The second opening 15′ at the side face in the bottom portion of the container 1′ can be used to empty the container 1′ during its use. This can be particularly advantageous if the container 1′ is filled with a liquid content. For example, a tube can be sealingly connected to the second opening 15′ for draining the liquid content in the container 1′.

FIG. 13a shows a perspective view of an embodiment of a container 1″ comprising a circumferential stabilization bead 16. The stabilization bead 16 is an outward protrusion of the wall of the container 1″ and is positioned next to the thread 12″. In general, the stabilization bead 16 is substantially thicker than the thickness of the container wall, having a substantial stabilization effect, for example increasing the torsional stability of the container 1″ when the thread 12″ is engaged with the base element (cf. FIG. 3). As may be easily seen in the embodiment according to FIG. 13a, the container 1″ comprises an inner neck 17. The inner neck 17 adjoins the circumferential collar 14′. The inner neck 17 may contribute to locking a cover element to the container 1″, as described above (cf. also FIG. 10).

FIG. 13b shows a side view of the container according to FIG. 13a.

FIG. 14a shows a perspective view of an embodiment of a base element 11′, comprising a circumferential outer side wall 111 and a circumferential inner side wall 112. The portion of the container adjacent to the second opening is receivable in the space between the outer side wall 111 and the inner side wall 112. The stability of the base element 11′ is increased by the shown double wall configuration comprising the outer side wall 111 and the inner side wall 112. The outer side wall 111 comprises as counter base locking means a counter thread 113 which is operatively interconnectable to the thread (cf. FIG. 3) at the portion of the container adjacent to the second opening. The inner side wall 112 is configured to improve the sealed closing of the second opening by the base element 11′.

FIG. 14b shows a side cut view of the base element 11′ according to FIG. 14a.

FIG. 15a shows a schematic illustration of the embodiment of the container 1 according to FIG. 1 in a side view. The protection collar 24 of the cover element 2 is shown in a normal working position where the collar 24 protrudes with a positive collar angle α with respect to a horizontal face E formed by the top plane surface of the container 1. In this shown configuration, the protection collar 24 provides protection of a hand of a user disposing sharps to the disposable container 1.

FIG. 15b shows the container 1 according to FIG. 15a with the protection collar 24 folded to a configuration with an inverted collar angle α′ with respect to the horizontal face E. The upper part of the container 1 which is covered by the folded protection collar 24 is shown with dashed lines. This shown configuration is particularly advantageous for nesting since it reduces the stacking height.

There may be special other embodiments where the container 1 has a very different shape. For example, the container may have an elliptical or a polygonal cross section. Combinations of elliptical, circular or polygonal cross sections may be possible. Other complex geometries may include indentations or other variations modifying the cross-section. Indentations or grooves may be formed in the container for stabilization or for better grip. Stabilization using indentations or grooves may be particularly advantageous for containers with thin walls, for example for the containers made of PET. The indentations or grooves may be arranged horizontally or vertically.

For the use of disposing syringe needles, it may be advantageous that the inner surface of the container is a smooth surface, such that the grooves or indentations may be formed only with respect to the outer face of the container walls.

In an embodiment, the length of the largest convex cross-sectional envelope curve in the distal half of the container with respect to the first opening is larger than the length of the smallest convex cross-sectional envelope curve of the proximal half of the container with respect to the first opening.

For example, the largest convex cross-sectional envelope curve of a circular truncated cone is the circle of the base of the cone. For a cross-section with indentations, the convex envelope curve may be the contour of said cross-section leaving out said indentations.

In general, the operative interconnection between the container and the cover element or between the container and the base element by the one of the at least one locking means may be a releasable interconnection or an unreleasable interconnection. For a releasable interconnection, it may be necessary to exert a minimum force to release the operative interconnection. A sufficient minimum force to release said operative interconnection may be advantageous for safety issues such that the container is not opened accidentally.

In an embodiment, the container is unreleasably interconnectable to the cover element by one of the at least one locking means and releasably interconnectable to the base element by one of the at least one locking means.

Injection-moulded sharps disposal containers typically have a wall thickness exceeding a thickness of about 1 mm or above. With a blow-moulded container made of PET according to an embodiment of the present invention, wall thicknesses below 1 mm may be possible while maintaining a sufficient puncture-resistance. Preferably, a wall thickness of the container according to the present invention between around 0.2 mm and around 0.8 mm may still provide sufficient puncture-resistance.

The requirements on a sufficient puncture-resistance for sharps disposal containers are defined for example in DIN EN ISO 23907. According to the conformity test TRBA 250 a sufficient puncture-resistance is given by a resistance to a puncture of 15 N at a needle diameter of 1.1 mm with a BL/LB polished thin section. Preferably, the container according to the present invention complies with said requirements.

### LIST OF REFERENCE SIGNS

1, 1′, 1″ Container
11, 11′ Base element
111 Outer side wall
112 Inner side wall
113 Counter thread
12, 12″ Thread
13, 13′ First opening
14, 14′ Collar
15, 15′ Second opening
16 Stabilization bead
17 Inner neck
2 Cover element
21 Cap element
211 Hole
212 Cord
213 Latch
22 Pin
23 Cord recess
24 Protection collar
25 Handle
26 Cover opening
27 Stripper elements
28 Neck
281 Pin
282 Recess
4 Locking ring
41 Bayonet joint element
E Top plane surface
α Collar angle
α′ Inverted collar angle

1. Disposable container, in particular for the disposal of sharp medical waste, comprising a first opening sealingly closable by a cover element, characterized in that
a) the container is manufactured by blow-moulding;
b) the largest cross-sectional diagonal of the container in the distal half with respect to the first opening is larger than the smallest cross-sectional diag-onal of the container in the proximal half with respect to the first opening;
c) the container comprises a second opening arranged opposite to the first opening or in the distal area with respect to the first opening, the second opening being sealingly closable by a base element.

2. Container according to claim 1, characterized in that the container comprises at least one locking means, adapted for locking the cover element and/or the base element to the container.

3. Container according to claim 1, characterized in that a first locking means is provided at the first opening, the first locking means comprising a locking ring, and a second locking means is provided at the second opening.

4. Container according to claim 1, characterized in that the container is unreleasably interconnectable with the cover element and/or the base element in a force-fit or form-fit manner.

5. Container according to claim 1, characterized in that the container comprises at least one circumferential stabilization bead.

6. Container according to claim 1, characterized in that the container comprises a base element, the second opening being closable by said base element, said base element comprising a circumferential outer side wall and a circumferential inner side wall wherein the portion of the container adjacent to the second opening is receivable in the space between the outer and inner side wall.

7. Container according to claim 1, characterized in that the container is at least partially puncture-resistant.

8. Cover element for a container according to claim 1, the cover element comprising a cover opening and an operatively interconnected cap element, the cover opening being sealingly closable by the cap element, the cover element being configured to sealingly close an opening of the container, characterized in that the cover element comprises an integrally formed circumferential hand protection collar protruding with a positive collar angle with respect to a horizontal face of the container.

9. Cover element according to claim 8, characterized in that the cover element is at least partially puncture-resistant.

10. Cover element according to claim 8, characterized in that the circumferential hand protection collar is foldable to a configuration with an inverted collar angle with respect to a horizontal face of the container.

11. Method of producing a container according to claim 1, characterized in that the container is produced by a blow-moulding process leaving the second opening open.

12. Method of producing a container according to claim 11, characterized in that in a second step the first opening is introduced by punching.
